(19) United States
(12) Patent Application Publication
(10) Pub. No.: US 2015/0092188 A1
(43) Pub. Date: Apr. 2, 2015

(54) Apparatus and Method

(71) Applicant: The University of Lincoln, Lincoln (GB)

(72) Inventors: Nigel Allinson, Lincoln (GB); Thalis Anaxagoras, Lincoln (GB)

(21) Appl. No.: 14/398,589

(22) PCT Filed: May 7, 2013
(86) PCT No.: PCT/GB2013/051176

§ 371 Date: Nov. 3, 2014

(30) Foreign Application Priority Data
May 4, 2012 (GB) 1207918.2

(51) Int. Cl. G01N 21/47; G01N 33/483; G01N 21/64

(52) U.S. Cl. CPC G01N 21/47; G01N 21/64; G01N 33/4833; G01N 2201/062
USPC 356/72; 356/432

(57) ABSTRACT

An apparatus comprises a substrate (110), the substrate having: a source (141) for generating electromagnetic radiation for illuminating an object; a detector (120) for detecting a response, such as scattered or fluorescent radiation, by the object to illumination by the electromagnetic radiation; and a least one conduit (150) formed through at least a portion of the substrate for transmission of electromagnetic radiation therethrough in a path from the source to the object or a path from the object to the detector, wherein the source and the detector are not in direct line of sight of one another.

### FIELD OF THE INVENTION

The present invention relates to an electronic imaging device and to a method of forming an image of a subject using an electronic device.

### BACKGROUND

In most imaging applications, and not just optical imaging applications, a source of light or other excitation is physically separate to the detecting element. A simple example would be an apparatus for copying slides, which consists of a slide illuminated from one side while on the other side, perhaps with an intervening lens system, an imager (camera) is provided.

The present inventors have recognised that in some applications there are advantages in not physically separating excitation and detection. Examples include the imaging of thin biological tissues either directly or by observing their fluorescence, and accurate recording of fingerprints. By combining excitation and detection in the same physical location then more efficient collection of excitation is possible and equipment can be made more compact and potentially at lower cost.

It is desirable to understand biological processes such as neural communications processes in which electrical signals are generated in biological material. Various techniques have been reported for detecting neural activity such as electrical pulses transmitted by neurons.

In one known technique an image of brain activity is acquired by capacitive coupling of an electrical probe placed in close proximity to a portion of the brain. Charge is collected by the probe, the amount being proportional to the coupling capacitance and inversely proportional to input and stray capacitances. This technique may be used to detect activity in the neuron but cannot perform stimulation of the neuron. This detection of the activity requires the realisation of micro/nano probes that can penetrate the neural tissue and reach the desired neuron.

In another technique an array of probes in the form of electrically conducting needles is placed in direct electrical contact with the brain. The probes establish a capacitive coupling between a pixel of a detector and the neuron allowing electrical activity to be detected.

It is desirable to provide improved apparatus for imaging biological process such as inter-neural communications.

### STATEMENT OF THE INVENTION

In one aspect of the invention there is provided apparatus comprising:
a substrate, the substrate having:
a source for generating an inspection medium for illuminating a subject object;
a detector for detecting a response by a subject object to illumination by the inspection medium; and
at least one conduit formed through at least a portion of the substrate for transmission of inspection medium therethrough, the conduit being arranged wherein the source and detector are not in direct line of sight of one another.

In an aspect of the invention there is provided apparatus comprising:
a substrate, the substrate having:
a source for generating an inspection medium for illuminating a subject object;
a detector for detecting a response by a subject object to illumination by the inspection medium; and
transmission means for transmitting inspection medium through at least a portion of the substrate in a path from the source to the subject object or a path from the subject object to the detector, the apparatus being arranged wherein the source and detector are not in direct line of sight of one another.

Thus the apparatus is arranged such that the source and detector are blocked from being in a direct line of sight of one another whilst allowing transmission of inspection medium by means of the transmission means. The transmission means may be defined by an interface between a wall of a hole (which may be referred to as an aperture, pipe or conduit) formed in the substrate and a second medium such as a transmission medium for transmitting the inspection medium. The second medium may be vacuum, gas such as air or an inert gas such as argon or nitrogen, or a material such as a gel, a glass, an oxide or any other suitable medium. Thus the aperture, pipe or conduit may be gas filled, solid filled, such as glass filled, gel filled, liquid filled or filled with any other suitable gas, solid, semi-solid, liquid or other medium.

The transmission means may comprise a conduit or duct for allowing inspection medium to pass therethrough or therealong.

In some embodiments the apparatus may be considered to collimate the inspection medium such that the source and detector are not in a direct line of sight of one another. That is, a substantially straight path for the propagation of inspection medium from the source to the detector is blocked by a portion of the apparatus. The blocking portion may be a portion of the apparatus defining a boundary between the transmission means and the substrate, such as a portion of the substrate defining a conduit through the substrate, a layer formed in contact with the substrate and defining the conduit, or other portion.

The conduit may be in the form of a via, for example a through-via, a blind via, for example a blind via having a source provided therein, or any other suitable arrangement.

In another aspect of the invention there is provided apparatus comprising:
a substrate, the substrate having:
a source for generating an inspection medium for illuminating a subject object;
a detector for detecting a response by a subject object to illumination by the inspection medium; and
at least one conduit formed through at least a portion of the substrate for transmission of inspection medium therethrough in a path from the source to the subject object or a path from the subject object to the detector, the conduit being arranged wherein the source and detector are not in direct line of sight of one another.

Embodiments of the invention have the advantage that the detector is not exposed directly to illumination from the source. Thus, in the case that the detector is arranged to detect radiation from the subject object, only radiation scattered by or emitted by the subject object is detected by the detector and not illumination that has passed from the source to the detector in a direct line of sight.

It is to be understood that with reference to some embodiments, the words ‘radiation’ and ‘illumination’ may be used interchangeably. Furthermore, it is to be understood that in some embodiments inspection medium passes through the at least one conduit in a path from the source to the subject object and not a path from the subject object to the detector. In some embodiments inspection medium passes through the at least one conduit in a path from the subject object to the detector and not from the source to the subject object. In some embodiments inspection medium passes through the at least one conduit in a path from the source to the subject object and from the subject object to the detector. In some embodiments inspection medium does not pass from the subject object to the detector; for example, in some embodiments the detector is arranged to detect electrical signals or other signals generated by a subject object rather than inspection medium.

The inspection medium may be any suitable inspection medium such as any suitable type of radiation, including electromagnetic radiation, of any suitable wavelength.

Embodiments of the invention have the advantage that a signal generated by the detector responsive to illumination of the subject by the inspection medium is not swamped by a signal generated by the detector responsive to direct illumination of the detector by the source, i.e. line of sight transmission of inspection medium from the source to the detector.

By way of example, in the case that the detector is intended to detect optical radiation the detector may be arranged to detect optical radiation scattered by or emitted by the subject and not directly transmitted from the source to the detector in a line of sight. This has the advantage that a signal generated by the detector responsive to illumination of the sample by the inspection medium is not swamped by a signal generated by the detector responsive to direct illumination of the detector by the source.

In embodiments of the invention where the detector is arranged to detect an electrical signal generated by the sample, the fact that the source is not in a direct line of sight of the detector reduces a risk that a signal generated by the detector responsive to the electrical signal generated by the sample is influenced by illumination of the detector by the source.

For example, a semiconductor device employed to detect the electrical response of a sample to illumination by means of optical radiation may itself generate a response if it is illuminated with optical radiation. In this case, providing the source and detector such that they are not in a direct line of sight of one another reduces a risk that a signal generated by the detector is influenced by radiation from the source incident thereon.

Embodiments of the invention have the advantage that because the source and detector are provided by the same substrate, improved device performance may be obtained. Furthermore, this allows imaging of samples from the same side of the sample as the source. It can be advantageous in some applications to perform photon-electrical stimulation of the sample from the same side as that from which the sample is imaged, i.e. the side that may be touching or in close proximity to the detector.

It is to be understood that the conduit may be a hollow conduit (such as a gas filled conduit or an evacuated conduit) or filled with one or more elements arranged to convey inspection medium such as one or more optical fibres or the like. Thus the conduit may provide a waveguide for guiding inspection medium. Other arrangements are also useful.

The conduit may be in the form of a pipe, channel, tube or duct for conveying inspection medium.

Optionally a path is provided for inspection medium to travel from a subject object to the detector, the detector being arranged to detect inspection medium scattered by or generated by a subject object.

Advantageously a path of inspection medium from the source to the detector via a subject object may be required to pass through the at least one conduit.

Alternatively or in addition the path of the inspection medium from a subject object to the detector may be required to pass through the at least one conduit.

Advantageously a path of the inspection medium from the source to a subject object may pass through the at least one conduit.

Advantageously the apparatus may comprise a probe member optically coupled to the source and operable to convey inspection medium from the source to a subject object.

Optionally the probe member may be electrically coupled to the detector, the detector being configured to detect an electrical signal generated by a subject object responsive to irradiation of a subject object by the inspection medium.

Advantageously the probe member may be configured for insertion into tissue.

Further advantageously the apparatus may comprise a plurality of probe members.

The apparatus may comprise at least one probe member of a first length and at least one probe member of a second length different from the first whereby the apparatus may convey inspection medium to different respective depths of a subject object.

The source may be provided at one selected from amongst a surface of the substrate and a location within the substrate.

The detector may be provided at one selected from amongst a surface of the substrate and a location within the substrate.

Alternatively the detector may be provided within the substrate.

The source may be provided within the substrate.

The source and detector may be provided at opposite surfaces of the substrate.

The detector and source may be provided at the same surface of the substrate.

Advantageously the substrate may comprise a plurality of optical conduits.

The apparatus may be operable to transmit inspection medium along each of the plurality of conduits.

The source may be operable to transmit inspection medium along a plurality of the conduits substantially simultaneously.

This feature has the advantage that the same source may be used to illuminate a subject object at a plurality of respective positions of a subject object.

The apparatus may be operable sequentially to transmit inspection medium along a selected one or more different respective conduits.

This feature has the advantage that a subject object may be stimulated by inspection medium at different respective locations at different respective times, allowing a response of different locations of the object to be determined without a measured response at one location affecting a measured response at another location.

Optionally the source is movable with respect to the substrate thereby to allow inspection medium to be transmitted along different respective conduits.

This feature has the advantage that a single source may be employed to transmit inspection medium along a plurality of conduits.

Advantageously the apparatus may comprise a plurality of sources.

Each of the plurality of conduits may have a respective source associated therewith.

The apparatus may comprise a plurality of detectors.

Each of the plurality of conduits may have a respective detector associated therewith.

The apparatus may comprise a light guide arranged to guide light from the at least one conduit towards a subject object.

Advantageously the light guide may comprise an optical fibre.

Further advantageously the light guide may comprise a plurality of optical fibres.

Advantageously the apparatus may be operable to expose a subject object to at least one selected from amongst an electric field and an electromagnetic field.

Further advantageously the apparatus may comprise at least one conducting electrode between the substrate and a subject object by means of which the sample may be exposed to the at least one field.

Still further advantageously the apparatus may comprise a signal generator for generating a signal for application to the at least one conducting electrode.

Optionally the at least one conducting electrode is arranged to be provided between the substrate and a subject object.

The at least one electrode may be provided between the substrate and the light guide.

Alternatively the at least one electrode may be arranged to be provided between the light guide and a subject object.

Optionally the at least one electrode is arranged to be coupled to a power supply by means of one or more signal lines passing along a conduit.

The apparatus may comprise a display panel, the apparatus being operable to transmit inspection medium through the display panel, the detector being operable to detect a subject object beyond the display panel.

Thus the apparatus may be operable to provide for example touch screen functionality.

The display panel may comprise an electronic display panel.

The display panel may for example be a liquid crystal display (LCD) panel, a light emitting diode (LED) panel, a plasma display panel or any other suitable panel.

The apparatus may be operable to form an image of a subject object.

Thus in some arrangements the apparatus may be employed in an object image recording or object recognition application.

In a further aspect of the invention there is provided a detector module comprising apparatus according to the first aspect.

In an aspect of the invention there is provided a system comprising apparatus according to a preceding aspect, the system being operable to generate an image of a subject object by detection of a response by a subject object to illumination by the inspection medium.

In one aspect of the invention there is provided a method comprising the steps of:
generating by means of a source of a substrate an inspection medium for illuminating a subject object; and
detecting by means of a detector of the substrate a response by the object to illumination by the inspection medium,
the method further comprising stopping inspection medium generated by the source from irradiating directly the detector by causing the inspection medium to pass through an optical conduit formed through at least a portion of the substrate as the inspection medium travels from the source to the subject object or from the subject object to the detector.

In an aspect of the invention there is provided a method comprising the steps of:
generating by means of a source of a substrate an inspection medium for illuminating a subject object; and
detecting by means of a detector of the substrate a response by the object to illumination by the inspection medium,
the method further comprising stopping inspection medium generated by the source from irradiating directly the detector by causing the inspection medium to pass through an optical conduit formed through at least a portion of the substrate.

In a further aspect of the invention there is provided apparatus comprising:
a substrate, the substrate having:
a source for generating an inspection medium for illuminating a subject object;
a detector for detecting a response by an object to illumination by the inspection medium; and
at least one conduit formed through at least a portion of the substrate for transmission of inspection medium from the source to an object or from an object to the detector, the conduit being arranged wherein the source and detector are not in direct line of sight of one another.

Thus in some arrangements the at least one conduit allows passage of inspection medium from the source to a subject object. In some arrangements the at least one conduit allows passage of inspection medium from a subject object to a detector. The at least one conduit, the source and the detector are positioned such that the source and detector are not in a direct line of sight of one another. Thus, a sidewall of the conduit is provided in a direct line between the source and the detector such that the source and detector are not in a direct line of sight. It is to be understood that the substrate may be at least partially transparent to the inspection medium which may be optical radiation such as visible light, infra-red light, ultra violet light or any other kind of radiation. However the presence of the sidewall and/or at least a portion of the substrate in the direct line of sight between source and detector reduces an amount of inspection medium that can travel in a direct line from the source to the detector, reducing a risk that

In an aspect of the invention there is provided apparatus comprising:
a substrate;
source for generating optical radiation or other inspection medium for illuminating a subject object; and
detector for detecting response by the object to the optical radiation,
wherein the source and detector are provided in or on a common substrate, the substrate having at least one optical conduit for optical radiation formed through at least a portion thereof, a sidewall of the conduit being arranged to block optical radiation such that the source and detector are not in direct line of sight of one another.

The side wall of the conduit may be shielded with a material and/or the conduit may have a light conveying medium provided therein for example in the form of a tube or pipe, whether hollow or solid. For example one or more optical fibres may be provided within the conduit. The light conveying medium may be arranged to pass an optical signal therethrough for optically stimulating a subject object without affecting the detection behaviour of the apparatus to a signal generated by a subject object in response to stimulation.

In one aspect of the invention there is provided an imaging device comprising:
a source of optical radiation for illuminating a subject; and
a corresponding detector for detecting optical radiation from the subject,
the source and detector being formed in or on a common substrate, the substrate having a hole through at least a portion thereof arranged wherein an optical path from the source to the detector via the subject is required to pass through the hole, the source and detector being arranged wherein they are not in direct line of sight of one another.

Another possible application of embodiments of the invention is to be able realise a display on the surface of an imaging device. This technique allows the realisation of a display underneath the imaging device (or ‘imager’) or an imager underneath a display.

In the case the imager is below the display, the conduits may be required to pass optical radiation or another inspection medium through the display itself.

### BRIEF DESCRIPTION OF THE DRAWINGS

Embodiments of the invention will now be described with reference to the accompanying figures in which:

FIG. 1 is a schematic illustration of a structure according to an embodiment of the invention;

FIG. 2 is a schematic illustration of a touch-screen display module according to an embodiment of the invention;

FIG. 3 is a schematic illustration of a structure according to a further embodiment of the invention;

FIG. 4 shows a probe of the structure of the embodiment of FIG. 3;

FIG. 5 shows (a) a sensor module according to an embodiment of the invention in cross-section prior to insertion into brain tissue; (b) the sensor-module of (a) in plan view; and (c) a cross-section of a portion of a sensor module;

FIG. 6 is a cross-sectional view of a sensor module according to a further embodiment of the invention;

FIG. 7 is a plan view of a portion of the module of FIG. 1;

FIG. 8 is a plan view of a portion of a module according to a further embodiment of the invention;

FIG. 9 is a plan view of a module according to an embodiment of the present invention; and

FIG. 10 is a plan view of a module according to a further embodiment of the invention.

### DETAILED DESCRIPTION

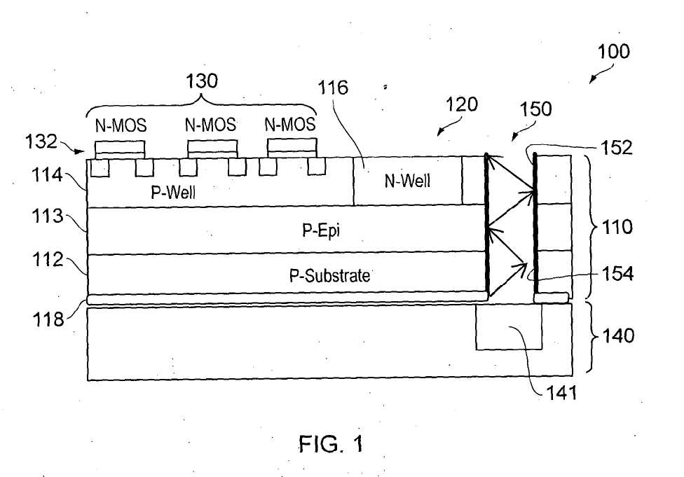

FIG. 1 shows an optical detector structure 100 according to an embodiment of the present invention. The structure 100 has an optical illumination source 140 and an optical detector device 120 for detecting optical radiation emitted by the source 140. In some embodiments a source is provide arranged to generate an inspection medium other than optical illumination such as electromagnetic radiation of a different wavelength. That is, photons of any suitable wavelength may be employed. A source of other types of particle other than photons may be employed in some embodiments.

The structure 100 includes a substrate 110 formed from a p-type doped silicon wafer 112 over which a first epitaxial layer 113 of p-type doped silicon has been formed. A second layer 114 of p-type doped silicon has been formed over the first layer 113. On an opposite (rear) side of the wafer 112 a conducting layer of a metallic material has been formed, providing a back contact 118 by means of which an electrical connection may be made to the substrate 110. The contact 118 is arranged to shield the rear side of the substrate 112 from any incident illumination. In some arrangements a shield layer is provided instead that is not arranged to provide an electrical contact. The shield layer may be formed from any suitable material, including any suitable insulating, semiconducting or conducting material. Other materials are also useful.

It is to be understood that other forms of silicon substrate 110 may be employed in addition or instead, such as doped or undoped substrates 110. Other materials are also useful including other semiconductor substrates. Insulating substrates may be useful in some embodiments.

The second layer 114 has a region of n-type doped silicon 116 formed therein, the n-type doped silicon region 116 being formed over and in contact with the first layer whereby a photodiode device 120 is formed by the n-type doped region 116 and the first layer 113.

An electrical drive circuit 130 is formed in the second layer 114 adjacent the photodiode 120. The drive circuit 130 includes N-MOS transistor devices 131 as shown in FIG. 1. The circuit 130 is configured to apply a reverse bias potential to the photodiode 120. It is to be understood that the (in-pixel) drive circuit 130 may be of any suitable form. For example, other types of transistor device may be employed.

An illumination panel 140 is provided below the back contact 118. The illumination panel 140 provides a source of optical illumination for a sample to be inspected by means of the structure 100.

In the embodiment shown the panel 140 includes a light emitting diode (LED) device 141 operable to project optical radiation through an optical conduit 150 formed through the substrate 110. The structure 100 is thereby operable to illuminate a sample of material to be inspected that is provided in a path of radiation generated by the LED device 141.

The optical conduit 150 has a sidewall 152 having an optical anti-reflection coating 154 formed thereover. The coating 154 is arranged to reduce an amount of optical radiation reflected from the sidewall 152 back into the conduit 150. In some alternative arrangements the coating 154 may be a reflecting coating arranged to reflect light incident thereon back into the conduit.

The apparatus 100 is arranged wherein the LED device 141 and photodiode 120 are not in a direct line of sight of one another due to the collimating effect of the optical conduit 150. That is, the sidewall 152 of the conduit 150 blocks light from the LED device 141 from irradiating directly the photodiode 120.

This feature is advantageous in reducing a risk that an optical signal from a sample that is irradiated by the LED device 141 is swamped by a signal due to direct irradiation of the photodiode 120 with radiation from the LED device 141.

In some arrangements where a plurality of structures 100 are provided, LEDs of different respective structures 100 may be activated independently of one another, i.e. a single LED device 141 (or any other type of source) may be arranged to transmit light or other radiation along a single conduit 150.

In some applications it is important to illuminate only part of the detector with light emitted by or scattered by a sample, and not all of the detector. Accordingly a shield may be provided for shielding at least a portion of the detector from incident radiation.

In use, the structure 100 is positioned to irradiate a sample by means of the LED device 141. Radiation scattered by or emitted by the sample and falling on the photodiode 120 is then detected by the drive circuit 130, which monitors current flowing through the photodiode 120 via the back contact 118.

In some embodiments, a single LED device 141 is employed to irradiate the sample through a single conduit 150. In some alternative embodiments a plurality of LED devices 141 are arranged to illuminate a sample via respective conduits 150.

In some arrangements the structure 100 is arranged wherein only a portion of a photodiode 120 is illuminated by optical radiation from the sample, as described above.

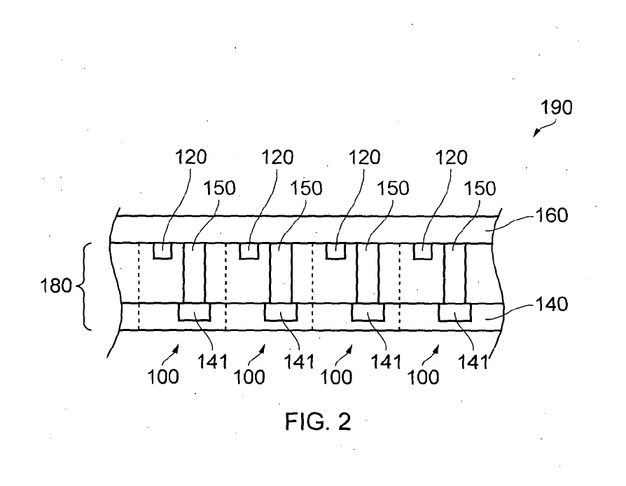

Embodiments of the invention are useful in a range of applications. For example, in some embodiments a touch-screen display module 190 is provided as shown in FIG. 2. The module 190 includes an array 180 of repeat units of the structure 100 of FIG. 1. The array 180 is employed to provide illumination of a liquid crystal diode (LCD) panel 160 in addition to detector functionality for the module 190.

As shown in FIG. 2, the array 180 provides backlighting to the LCD panel 160, the LED diode devices 141 being controlled to illuminate the panel 160 from a side opposite that from which a user views the panel 160. It is to be understood that other arrangements are also useful.

If a user touches the LCD panel 160 with an object such as a finger or a stylus, scattering of light due to contact between the object and the panel 160 causes a change in intensity of the illumination incident on the photodiodes 120. The change in intensity may be detected by a detector circuit (not shown) coupled to the photodiodes 120.

In some other embodiments an array of structures 100 may be arranged to provide illumination for respective pixels of a display module. For example respective LED devices 141 of an array of structures 100 may be arranged to generate white light illumination (for example by illuminating a phosphor material with light of a sufficiently low wavelength) and to illuminate red, green or blue filters or other colour emitting elements to provide a colour display module. Alternatively the devices 141 may be arranged to provide a monochrome display module. Still further alternatively, instead of generating white light, the LED devices 141 may be arranged to generate light of respective different wavelengths directly. For example certain devices 141 may generate red light, others green light, and others blue light. The requirement to provide filters may therefore be eliminated in some arrangements.

Again, the photodiodes 120 may provide means for detecting contact between an object and the display module.

It is to be understood that the apparatus 100 may be employed to form an image of an object. Modules in the form of 1D or 2D arrays of the structure 100 are particularly useful in imaging applications.

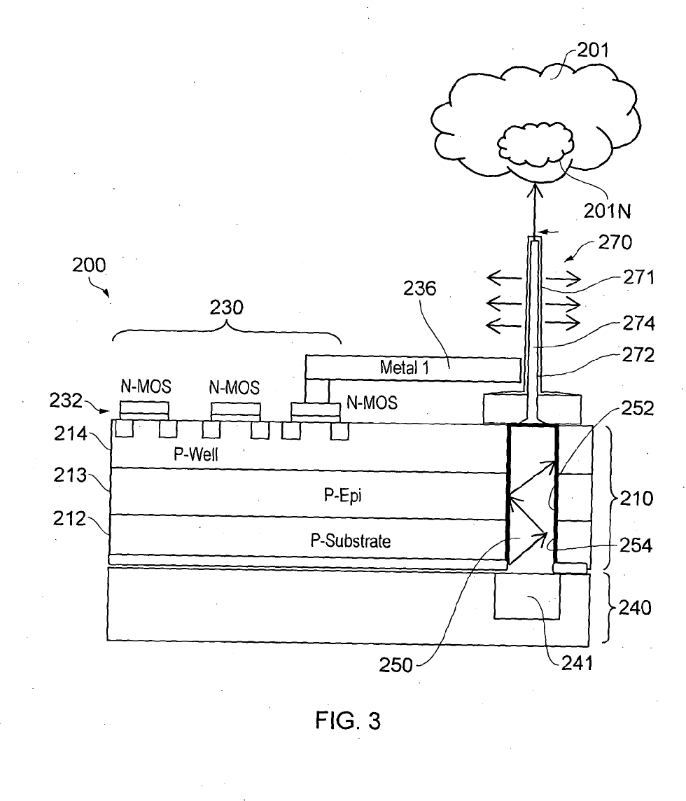

FIG. 3 shows a detector structure 200 according to a further embodiment of the invention. Like features of the embodiment of FIG. 3 to those of the embodiment of FIG. 1 are labelled with like reference signs prefixed numeral 2 instead of numeral 1.

The structure 200 includes a substrate 210 similar to that of the embodiment of FIG. 1. The substrate 210 is formed from a p-type doped silicon wafer 212 over which a first layer 213 of p-doped epitaxial silicon is formed, the first layer 213 having a second layer 214 of p-type doped silicon formed thereover.

The substrate 210 has an optical conduit 250 formed therethrough. An LED device 241 is arranged to project optical radiation through the conduit 250. A probe member 270 is coupled to the substrate 210 at a free surface of the substrate 210 opposite that at which the LED device 241 is provided.

The probe member 270 provides a further optical conduit and is arranged to convey optical radiation emerging from the conduit 250 along the length of the probe member 270.

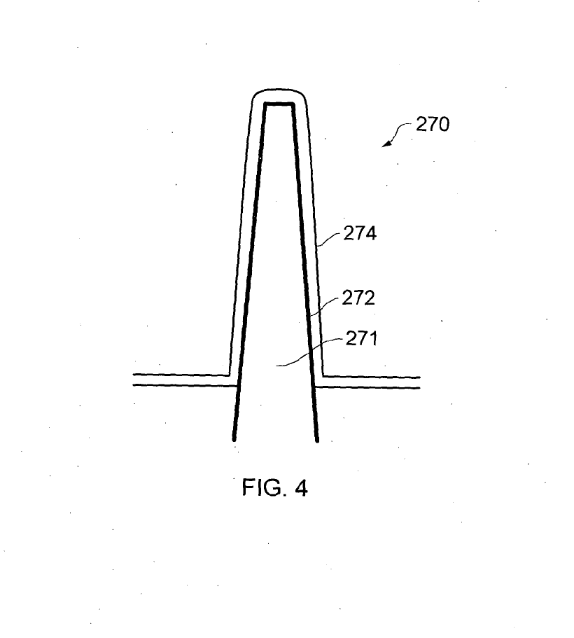

As shown in FIG. 4, the probe member 270 is in the form of a hollow silicon pipe element 271 having a layer 272 of a conducting material formed over it. An insulating layer 274 is formed over the conducting layer 272. In some embodiments the conducting layer 272 is formed from a metallic material such as nickel. In some embodiments the conducting layer may be a conducting oxide layer such as an indium tin oxide (ITO) layer. Other materials are also useful. The insulating layer 274 may be formed from an oxide layer such as silicon oxide, silicon nitride or any other suitable material.

In the embodiment shown, the probe member 270 is arranged whereby light entering the pipe element 271 from the conduit 250 is scattered out from the probe member 270 through the conducting and insulating layers 272, 274 along substantially the whole length of the probe member 270. In some embodiments the probe member 270 is arranged whereby optical illumination can only exit the probe member 270 along a prescribed portion of a length thereof, for example a portion towards a free end of the probe member 270. This is to allow the probe member 270 to illuminate a required portion of a sample to be investigated.

The conducting layer 272 is coupled by means of a conducting strap element 236 to an input stage of a detector circuit 230 formed in the second layer 214 of the substrate 210.

In use, the probe 270 is inserted into an object to be investigated, which may for example be brain tissue 201 of a subject as illustrated schematically in FIG. 3. It is to be understood that the strap element 236 then becomes coupled capacitively to the brain tissue 201 by means of the conducting layer 272 and insulating layer 274.

Optical illumination is directed along the probe member 270 and an electrical response by the brain tissue 201 to the illumination is detected by means of the detector circuit 230 via the capacitive coupling of the strap element 236 to the tissue.

In some arrangements illumination of a neuron 201N in the brain tissue 201 by the probe 270 results in stimulation of the neuron 201N and generation by the neuron 201N of a pulse of electrical potential. The pulse may be detected by the detector circuit 230 as described above.

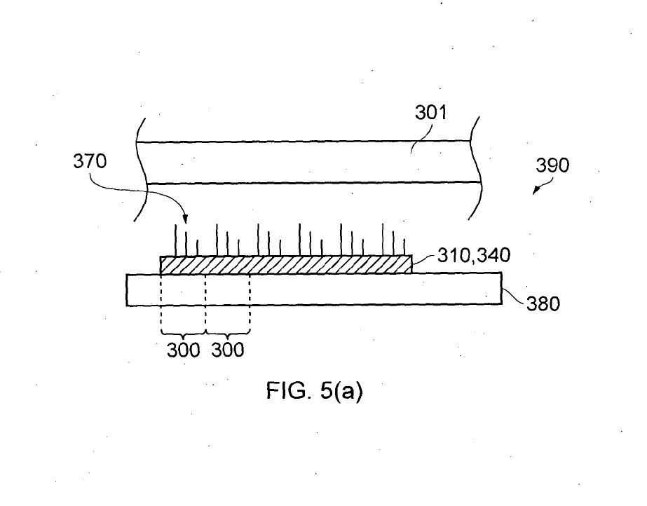

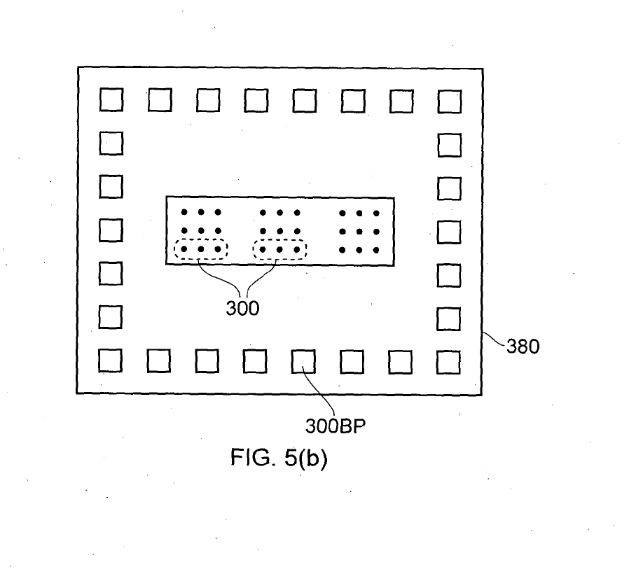

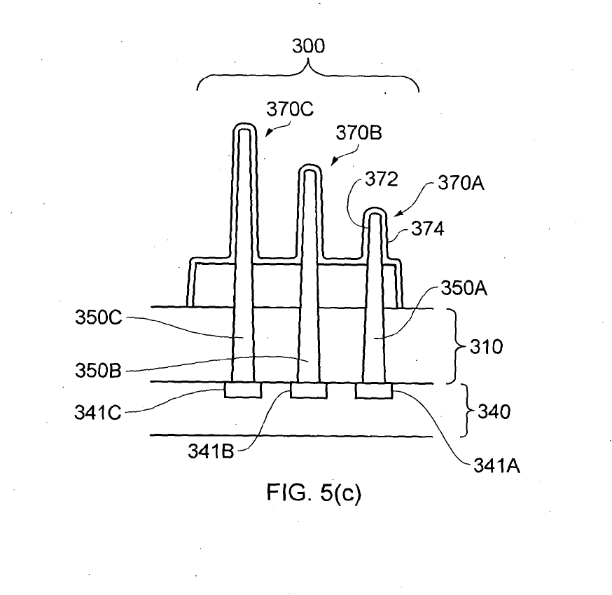

FIG. 5(a) shows a sensor module 390 according to an embodiment of the present invention in side view, together with a portion of brain tissue 301. FIG. 5(b) shows the module 390 in plan view. The module 390 has a 2D array of detector structures 300 (FIG. 5(b)) provided on a die 380. FIG. 5(c) shows one of the detector structures 300 in cross-section. Like features of the embodiment of FIG. 5 to those of the embodiments of FIG. 1, FIG. 2 or FIG. 3 are labelled with like reference signs prefixed numeral 3 instead of numeral 1 or numeral 2.

The die 380 has bond pads 380BP allowing electrical connection to be made so that signals generated by the structures 300 may be read out from the module 390. As shown in FIG. 5(c), each of the structures 300 is formed from three of the structures 200 of the embodiment of FIG. 3. The structures 300 each have three probe members 370 labelled respectively 370A, 370B, 370C in FIG. 5(c). Each of the probe members 370A, 370B, 370C of a given structure 300 is of a different length to the other probe members of that structure 300. By way of illustration FIG. 5(c) shows the probe members 370A, 370B, 370C of one repeat unit in cross-section.

The provision of probes of different respective lengths allows the module 390 to detect electrical activity at different respective depths of brain tissue 201 (or any other sample) into which the probes 370 are inserted. In some arrangements electrical activity can be monitored at different depths by respective probes of the module substantially simultaneously. By providing a 2D array of structures 300, electrical activity at different respective depths of a sample may be monitored substantially simultaneously across a given 2D area, enabling investigation of activity in a given 3D volume of a sample to be monitored. Some embodiments of the present invention permit unprecedented monitoring of electrical activity in a biological (or other) sample in real time. Some embodiments are suitable for monitoring activity in living patients without removal of an organ to be sampled from the body, for example in-situ monitoring of brain activity, nerve activity or activity in any other portion of a patient into which the probes 370 may be introduced.

FIG. 5(a) shows a sensor module 390 immediately prior to insertion of probes 370A, 370B, 370C thereof into brain tissue 301.

It is to be understood that in the arrangement described, the probe member 370A, 370B, 370C delivering optical radiation to the sample also detects the electrical response of the sample to the optical radiation. In some alternative arrangements, electrical activity stimulated in a sample by irradiation of the sample with radiation from one probe member may be detected by a different probe member, for example an adjacent probe member. Other arrangements are also useful.

It is to be understood that embodiments of the invention such as those illustrated herein have the feature that a single integrated source/detector apparatus may be provided. Thus the source and detector may be integrated together by means of a single substrate, which in some embodiments is a semiconductor substrate. That is, the source and detector may be formed in or on a single semiconductor substrate. It is to be un

It is to be understood that some source/detector arrangements such as those illustrated may be referred to as a single ‘device’ in the sense that the source and detector are integrated together by means of a single substrate.

As shown in FIG. 5 (a) the structure 300 is provided on a die 380 bearing circuitry that is arranged to process signals detected by the structures 300. FIG. 5(a) shows the module 390 at a stage of a process in which the probe members 370 are being introduced into a nerve sample 301.

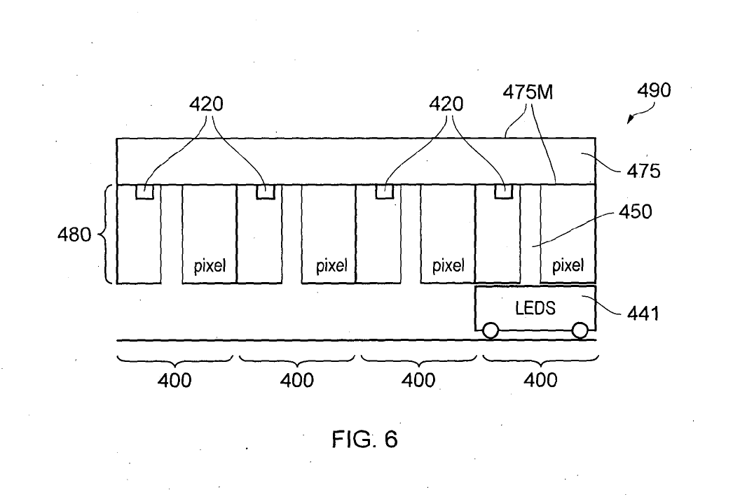

FIG. 6 shows a sensor module 490 according to a further embodiment of the invention. The module 490 is similar to that of the embodiment of FIG. 2 and includes an array 480 of repeat units of a structure 400 similar to the structure 100 of FIG. 1. The structure 400 differs from that of FIG. 1 in that the source of optical illumination from the structures 400 is a movable source in the form of a movable LED device 441. Thus the structures 400 share a single, common LED device 441 which provides illumination to respective structures 400 sequentially as the device 441 is moved from one end of the array 480 to the other. Whilst a single column (1D array) of structures 400 is shown in FIG. 6, it is to be understood that a 2D array may alternatively be provided. In embodiments having a 2D array of structures 400 the structures 400 may be illuminated by a 1D array of LED devices 441, by a single device 441, or a 2D arrangement of devices 441. Other arrangements are also useful.

The module 490 is provided with a fibre optic element 475 over the array 480. The element 475 has a bundle of optical fibres oriented normal to opposed (upper and lower) major surfaces 475M of the element 475 and arranged to convey light between the surfaces 475M.

The presence of the element 475 has the advantage that in some embodiments damage to the array 480, due for example to abrasion or contact with one or more chemicals, may be prevented. In some embodiments the fibre optic element provides a surface with which a sample (subject object) may be brought into contact in order to inspect the sample.

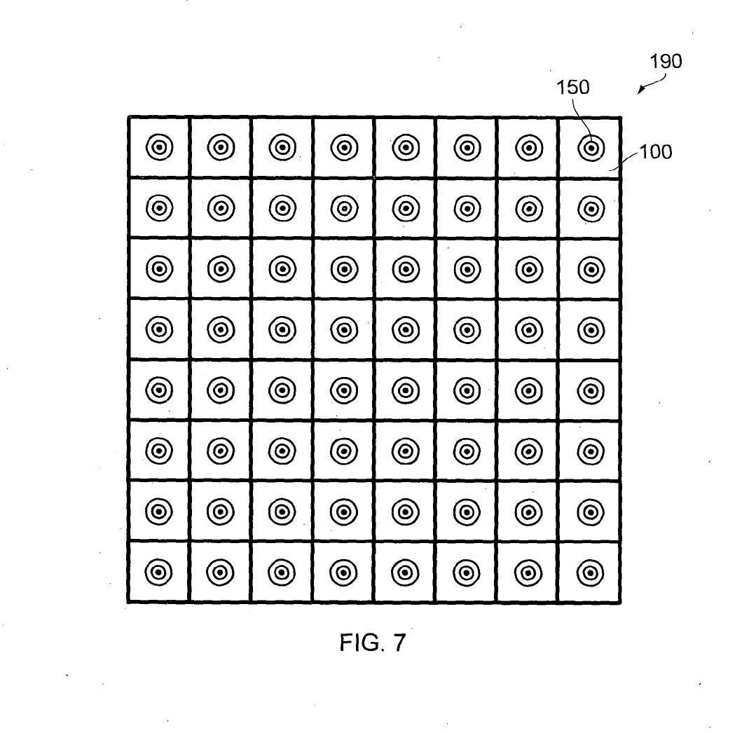

FIG. 7 shows the touch-screen LCD display module 190 of FIG. 2 in plan view with the LCD panel 160 removed. The It can be seen that each of the structures 100 of the module 190 has a dedicated optical conduit 150 for illuminating the panel.

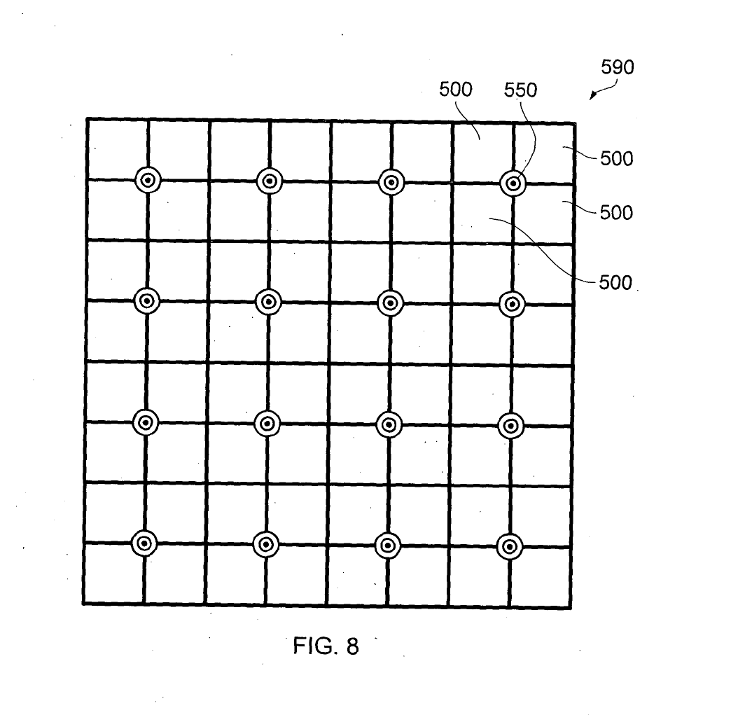

FIG. 8 shows a touch-screen LCD display module 590 according to an alternative embodiment of the invention also with the corresponding LCD panel removed. In this embodiment, a 2D array of structures 500 is provided. The structures 500 are similar to the structures 100 of FIG. 1 except that the structures 500 are arranged whereby groups of four immediately adjacent structures 500 provided in a square arrangement share a corner at a common point at which a single common optical conduit 550 is provided. In the embodiment shown, the conduit 550 is formed substantially at the common point of each group four structures 500. This is in contrast to the arrangement of FIG. 2 where each structure 100 has its own respective conduit 150.

This feature has the advantage that a reduced number of conduits 150 is required for a given number of photodiode detector devices 120. In some embodiments a corresponding decrease in the number of LED devices 141 required is also possible. Some embodiments have the advantage that the photodiode devices may be made to have a larger area since the amount of real estate occupied by conduits may be reduced.

It is to be understood that in some embodiments of the present invention other semiconductor substrate materials are also useful in addition to or instead of silicon. For example, GaAs, GaN or any other suitable semiconductor material may be employed. Other Group IV, III-V or II-VI materials are also useful.

In some alternative embodiments the substrate may be formed from an insulating material such as a glass material, an oxide material such as alumina or sapphire or any other suitable insulating material.

It is to be understood that embodiments of the invention may be operated in a variety of different ways. In some embodiments, detection of a response by an object to illumination is performed whilst the object is being illuminated, whether by means of a photodetector or a probe capacitively coupled to the object. In some alternative embodiments detection of a response is performed after illumination has been terminated.

For example, in some embodiments light emitted by an object by fluorescence is detected after terminating illumination of the sample.

Similarly, in some embodiments electrical activity in a subject object may be monitored during and following optical stimulation of the object.

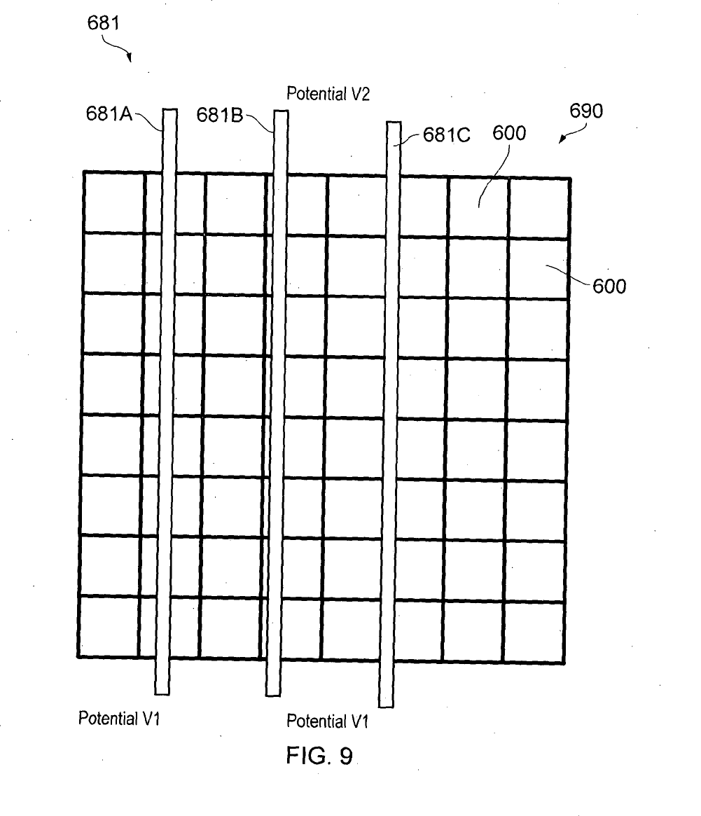

FIG. 9 shows a sensor module 690 according to a further embodiment of the invention. Like features of the embodiment of FIG. 9 to those of the embodiment of FIG. 1 are labelled with like reference signs prefixed numeral 6 instead of numeral 1.

The module 690 has a 2D array of structures 600 similar the structures 100 of the embodiment of FIG. 1. An array 681 of parallel electrodes 681A, 681B, 681C is provided over the structures 600. The array 681 is coupled to a power source operable to apply an electrical potential to the array 681. The presence of the array 681 allows an electric field to be applied to a sample by application of an electric potential to the array 681.

In some embodiments the module 690 is operable to induce an electromagnetic field in a sample by applying a time-varying electric potential to the array 681.

It is to be understood that the ability to apply an electric or electromagnetic field to a sample allows a response of the sample to such a field to be studied. In some samples, a state or configuration of the sample may change responsive to application of a field. A response of the sample to illumination with an inspection medium such as optical radiation may therefore be studied under different applied field conditions allowing a wide range of investigations to be performed by means of the apparatus.

Embodiments of the invention have the advantage that investigation of a sample may be performed in a convenient manner using an integrated detector device operable to illuminate a sample with inspection medium, and detect a response of the sample to illumination under different conditions of applied electric or electromagnetic field.

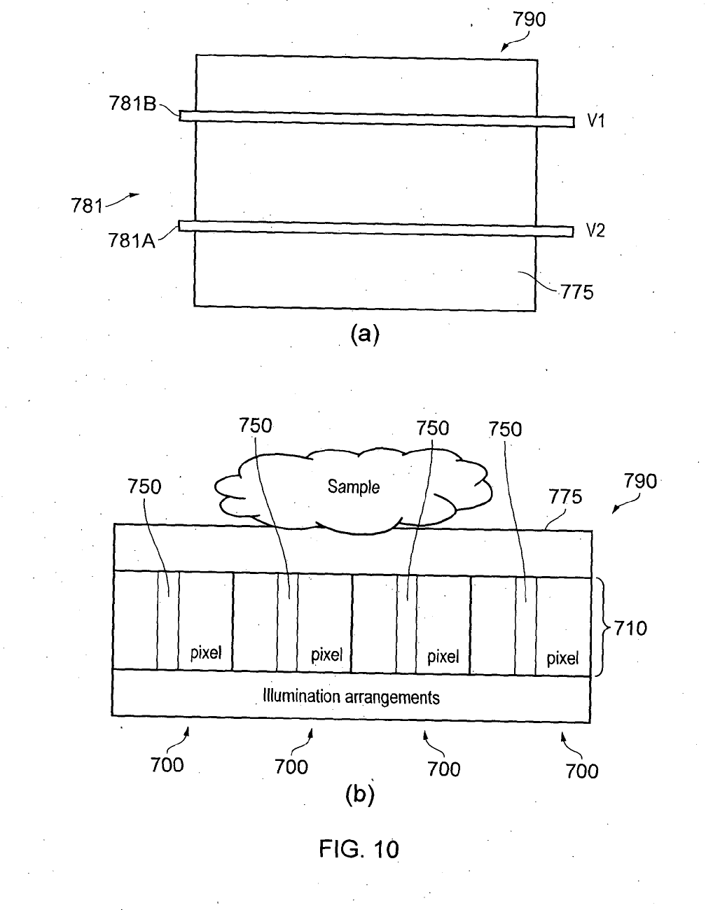

FIG. 10 shows a sensor module 790 according to a further embodiment of the invention. Like features of the embodiment of FIG. 10 to those of the embodiment of FIG. 9 are labelled with like reference signs prefixed numeral 7 instead of numeral 6.

As in the embodiment of FIG. 9 the sensor module 790 has a 2D array of structures 700 each of which is similar to the structure 100 of the embodiment of FIG. 1. A fibre optic coupling element 775 is provided over the structures 700 and an array 781 of parallel electrodes 781A, 781B is provided above the coupling element 775.

This embodiment has the advantage that the structures 700 are protected from damage or contamination due to the presence of the coupling element 775. In some embodiments the coupling element is arranged to be provided in direct contact with a sample under inspection, allowing improved optical coupling between the module 790 and a sample in some arrangements.

It is to be understood that in some embodiments electrical connection is made to the electrode array 781 by means of wires (not shown) that pass through conduits 750 in the substrate 710. Other arrangements are also useful.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

1. An apparatus comprising:
a substrate, the substrate having:
a source for generating an inspection medium for illuminating a subject object;
a detector for detecting a response by the subject object to illumination by the inspection medium; and
at least one optical conduit formed through at least a portion of the substrate for transmission of inspection medium therethrough in a path from the source to the subject object or a path from the subject object to the detector, the conduit being arranged wherein the source and detector are not in direct line of sight of one another.

2. The apparatus of claim 1, wherein a path is provided for inspection medium to travel from the subject object to the detector, the detector being arranged to detect inspection medium scattered by or generated by the subject object.

3. The apparatus of claim 2, wherein a path of inspection medium from the source to the detector via the subject object is required to pass through the at least one conduit, and/or wherein the path of the inspection medium from the subject object to the detector is required to pass through the at least one conduit.

4. (canceled)

5. The apparatus of claim 1, wherein a path of the inspection medium from the source to the subject object passes through the at least one conduit.

6. The apparatus of claim 1, comprising at least one probe member optically coupled to the source and operable to convey inspection medium from the source to the subject object.

7. The apparatus of claim 6, wherein the probe member is electrically coupled to the detector, the detector being configured to detect an electrical signal generated by the subject object responsive to irradiation of the subject object by the inspection medium.

8. The apparatus of claim 6, wherein the probe member is configured for insertion into tissue.

9. The apparatus of claim 6, wherein the at least one probe member is a plurality of probe members.

10. The apparatus of claim 9, comprising at least one probe member of a first length and at least one probe member of a second length different from the first length, whereby the apparatus may convey inspection medium to different respective depths of the subject object.

11. The apparatus of claim 1, wherein the source is provided at one selected from amongst a surface of the substrate and a location within the substrate, and the detector is provided at one selected from amongst a surface of the substrate and a location within the substrate.

12-14. (canceled)

15. The apparatus of claim 1, wherein the source and detector are provided at opposite surfaces of the substrate or wherein the source and detector are provided at the same surface of the substrate.

16. (canceled)

17. The apparatus of claim 1, wherein the substrate comprises a plurality of optical conduits.

18. The apparatus of claim 17, operable to transmit inspection medium along each of the plurality of conduits.

19. The apparatus of claim 18, wherein the source is operable to transmit inspection medium along the plurality of conduits substantially simultaneously and/or sequentially to transmit inspection medium along a selected one or more different respective conduits, and/or the source is movable with respect to the substrate thereby to allow inspection medium to be transmitted along different respective conduits.

20-21. (canceled)

22. The apparatus of claim 17, comprising a plurality of sources and wherein each of the plurality of optical conduits has a respective source associated therewith.

23. (canceled)

24. The apparatus of claim 1, wherein the detector is provided as a plurality of detectors.

25. The apparatus of claim 24, wherein the at least one optical conduit is a plurality of optical conduits, wherein each of the plurality of conduits has a respective detector associated therewith.

26. The apparatus of claim 1, comprising a light guide arranged to guide light from the at least one conduit towards the subject object.

27. The apparatus of claim 26, wherein the light guide comprises at least one optical fibre.

28. (canceled)

29. The apparatus of claim 1, wherein the source is operable to expose a subject object to at least one selected from amongst an electric field and an electromagnetic field.

30. The apparatus of claim 29, comprising at least one conducting electrode between the substrate and the subject object, wherein the at least one conducting electrode is configured to expose the subject object to the at least one electric field and/or at least one electromagnetic field.

31. The apparatus of claim 30, comprising a signal generator that generates a signal for application to the at least one conducting electrode.

32. The apparatus of claim 30, wherein the at least one conducting electrode is arranged to be provided between the substrate and the subject object or between the substrate and a light guide of the apparatus, the light guide being arranged to guide light from the at least one conduit towards the subject object.

33. (canceled)

34. The apparatus of claim 32, comprising a light guide arranged to guide light from the at least one conduit towards the subject object, wherein the at least one electrode is arranged to be provided between the light guide and the subject object.

35. The apparatus of claim 30, wherein the at least one electrode is arranged to be coupled to a power supply via one or more signal lines passing along a conduit.

36. The apparatus of claim 1, comprising a display panel, the apparatus being operable to transmit inspection medium through the display panel, the detector being operable to detect the subject object beyond the display panel.

37. (canceled)

38. A detector module comprising the apparatus of claim 1.

39. A system comprising the apparatus of claim 1, operable to generate an image of the subject object by detection of a response by the subject object to illumination by the inspection medium.

40. A method, comprising:
generating, via a source of a substrate, an inspection medium for illuminating a subject object; and
detecting, via a detector of the substrate, a response by the subject object to illumination by the inspection medium; and
stopping the inspection medium from directly irradiating the detector by causing the inspection medium to pass through an optical conduit formed through at least a portion of the substrate as the inspection medium travels from the source to the subject object or from the subject object to the detector.

41. (canceled)
